(19) United States
(12) Patent Application Publication
(10) Pub. No.: US 2004/0170983 A1
(43) Pub. Date: Sep. 2, 2004

(54) Methods of determining a chemotherapuetic regimin based on loss of heterozygosity at the thymidylate synthase locus

(76) Inventor: Kathleen D. Danenberg, Altadena, CA (US)

Correspondence Address: KENYON & KENYON, 1500 K STREET, N.W., SUITE 700, WASHINGTON, DC, 20005, US

(21) Appl. No.: 10/373,752

(22) Filed: Feb. 27, 2003

(51) Int. Cl. C12Q 1/68; G06F 19/00; G01N 33/48; G01N 33/50

(52) U.S. Cl. 435/6; 702/20

(57) ABSTRACT

The present invention is directed to a method of predicting a response to a chemotherapeutic regimen based on loss of heterozygosity at the thymidylate synthase locus in cancer tissue. This method comprises determining normal tissue genotype for thymidylate synthase of a patient; determining tumor tissue genotype for thymidylate synthase of said patient; comparing the normal tissue genotype with the tumor tissue genotype; determining whether a loss of heterozygosity at the thymidylate synthase locus has occurred in the tumor tissue based on the comparison of the genotypes; and predicting a response to the chemotherapeutic regimen based on the loss of heterozygosity in the tumor sample.

### BACKGROUND OF THE INVENTION

Thymdiylate synthase (TS) catalyzes the reductive methylation of 2′-deoxyuridylate 5,10-methylenetetrahydrofolate to form 2′-thymidylate and dihydrofolate. This is an essential step in DNA biosynthesis. Since TS is the only de novo source of the thymine base and its reaction is one of the rate-limiting steps in DNA synthesis, inhibition of TS has been a fruitful approach in cancer chemotherapy (Danenberg, P. V., Biochim. Biophys. Acta, 473: 73-92, 1977). TS is the target enzyme for 5-fluorouracil (5-FU), which for almost 50 years have been one of the mainstay drugs for treatment of many cancers. 5-FU exerts its cytotoxic effect through TS inhibition by forming a stable ternary complex among 5,10-methylenetetrahydrofolate, TS and 5-fluoro-2″deoxyuridylate, the active metabolite of 5-FU. Since the appearance of 5-FU, other fluoropyrimidine-base therapies such as FUdR, UFT, S-1 and capecitabine as well as folate-based TS inhibitors such as ratitrexed, pemetrexed and nolatrexed have been developed. In vitro studies have shown that cells become resistant to TS inhibitors by up-regulating TS expression and raising intracellular TS levels (Wang, W., et al., Cancer Res., 61: 55055510, 2001), leading to the expectation that the amount of TS in tumors might be a predictor of response to TS-targeted therapy. Indeed, recent studies have shown that the TS expression does vary considerably among tumors and that sensitivity of various tumors to 5-FU-based chemotherapy is correlated with the intratumoral level of TS (Huang, C. L., et al., Int. J. Oncol., 17: 47-54, 2000; Nishimura, R., et al., Anticancer Res., 19: 5621-5626, 1999; Salonga, D., et al., Clin. Cancer Res., 6: 1322-1327, 2000; Shirota, Y., et al., J. Clin. Oncol., 19: 4298-4304, 2001; Yeh, K. H., et al., Cancer, 82: 1626-1631, 1998.) Moreover, high TS levels in tumors have also been shown to be associated with worse prognosis (Kralovanszky, J., et al., Oncology, 62: 167174, 2002; Nakagawa, T., et al., Lung Cancer, 35: 165-70, 2002).

The mechanisms by which TS expression is regulated in vivo have not yet been clearly defined. However, it has been suggested that polymorphisms occurring in the TS promoter might constitute one regulatory factor. The TS gene is known to have a unique 28 base pair tandemly repeated sequence in the 5′-untranslated region (5′-UTR) and is polymorphic in the numbers of this repeat (Horie, N., et al., Cell Struct. Funct., 20:191-197,1995). Most individuals have either a double tandem repeat (2R/2R), three repeat (3R/3R) or a heterozygous (2R/3R) genotype, although higher order repeats are found in a few cases (Marsh, S., et al., Genomics, 58: 310-312, 1999). The TS enhancer region (TSER) polymorphism is a partial determinant of TS protein expression in human gastrointestinal cancers (Kawakami, K., et al., Anticancer Res., 19: 3249-3252, 1999), which reference is herein incorporated by reference in its entirety.

Cancer tissue with 3R/3R genotype has showed significantly higher TS protein expression than that with 2R/3R genotype, which has been confirmed with in vitro experiments (Kawakami, K., et al., Clin. Cancer Res., 7: 4096-4101, 2001.), which reference is herein incorporated by reference in its entirety. This association between TS genotype and TS expression, together with the role of TS expression in a 5-FU-based chemotherapy, suggests that the TS genotype with respect to the number of tandem repeats might be at least a partial predictor for 5-FU-based chemotherapy. Indeed, several recent clinical correlative studies have obtained preliminary evidence that the TS genotype of this TSER is associated with response and survival of colorectal cancer patients treated with 5-FU-based therapies (lacopetta, B., et al., Br. J. Cancer, 85: 827-830, 2001; Marsh, S., et al., Int. J. Oncol., 19: 383-386, 2001; Pullarkat, S. et al., Pharmacogenomics J, 1, 65-70, 2001; Villafranca, E., et al., J. Clin. Oncol., 19: 1779-1786, 2001).

The presence of variable numbers of a 28 base pair tandem repeat sequence in the TSER has drawn considerable interest recently because this is the only genomic lesion besides p53 mutation that may predict to some extent the clinical outcome of patients treated with 5-FU based therapy. All studies done to date agree that possession of the 2R/2R genotype has consistently been associated with greater clinical benefit than the 3R/3R genotype in 5-FU-treated patients (Iacopetta, B., et al., Br. J. Cancer, 85: 827-830, 2001; Pullarkat, S. T. et al., Pharmacogenomics J, 1, 65-70, 2001; Villafranca, E., et al., J. Clin. Oncol., 19: 1779-1786, 2001; Etienne M C, et al., J. Clin. Oncol. 20:2832-43, 2002). The discovery of TS polymorphisms that might be tumor response determinants was of considerable interest because of the possibility that prediction of tumor response could be done by analysis of readily available normal tissue (e.g, peripheral blood cells). This expectation is based on the assumption that the genotype in the normal tissue would be identical to that in cancer tissue. However, recently obtained evidence shows that this assumption is not always true in the case of TS genotype. A high incidence of LOH has been observed at the TS locus in cancer tissues, which leads to modification of TS genotype in the tumor when it is heterozygous in normal tissue (Kawakami K, et al., Jpn J Cancer Res. 93: 1221-1228, 2002; Zinzindohoue F, et al., J. Clin. Oncol. 19:3442, 2001). That is, the occurrence of LOH in individuals who have a heterozygous 2R/3R genotype in their normal tissue would give rise to a tumor with either a 2R/loss or the 3R/loss TSER genotype. Thus, it is possible that patients who are heterozygous and who have LOH at the TS locus in their tumor tissue might experience considerably different outcomes from chemotherapy depending on which allele became deleted during the LOH event.

Thus, there remains a need for a method to predict a response to various chemotherapuetic regimens prior to treatment. An accurate prediction will allow a physician to determine whether to go forward with a desired chemotherapeutic regimen or to try an alternative chemotherapeutic regimen. Since adverse side affects are prevalent with most chemotherapeutic regimens, it is desirable to be able to predict tumor response to the chemotherapeutic agent so as to eliminate any unnecessary or unsuccessful treatments. Further, if a physician is able to predict the response to a treatment, time will not be wasted on those treatments that are likely to fail and instead allow the physician to focus on more promising treatments. A method of predicting a response to treatment provides a physician with guidelines in choosing the therapy rather than a simple trial and error approach.

### SUMMARY OF THE INVENTION

The present invention is directed to a method of predicting a response to a chemotherapeutic regimen based on loss of heterozygosity at the thymidylate synthase locus in cancer tissue. This method comprises determining normal tissue genotype for thymidylate synthase of a patient; determining tumor tissue genotype for thymidylate synthase of said patient; comparing the normal tissue genotype with the tumor tissue genotype; determining whether a loss of heterozygosity at the thymidylate synthase locus has occurred in the tumor tissue based on the comparison of the genotypes; and predicting a response to the chemotherapeutic regimen based on the loss of heterozygosity in the tumor sample.

The present invention also contemplates utilizing the genotype of the normal tissue to help predict the patient's response (as in drug toxicity response) to the chemotherapeutic regimen.

To assist is predicting the responses, the present invention contemplates compiling patient data, such as a database, that references patient data such as, but not limited to, both normal tissue and cancer tissue genotypes, the cancer being treated, the chemotherapeutic regimen administered, the patient's response, i.e. tumor size reduction, if any, tumor growth, increase in metastases, patient survival, chemotherapeutic dose, toxicity data, life span of patient, etc. The data base will assist in predicting the response of a patient by allowing one to compare the patient's factors with previous patient's having like factors (such as normal tissue and cancer tissue genotype, chemotherapeutic regimens, and outcome of treatment).

In both the predicting a response as well as creating the database, fresh or preserved tissue may be used to determine the tissue genotype.

### BRIEF DESCRIPTION OF THE DRAWINGS

FIG. 1A shows the structure of TS 5′ flanking region with a 3-repeat sequence. The translated region is indicated by the solid bar. The open bar with arrows in it indicates the TSER. The arrows represent tandemly repeated sequences and a complementary reverse sequence. The numbers indicate nucleotide position when the first nucleotide of the initiation codon is defined to be +1. The PCR primers are designed to flank the region of the tandem repeats, so that the presence of 3 repeats will give a longer PCR product than a 2-repeat TSER.

FIG. 1B shows examples of TS genotype analysis in matched normal (N) and tumor (T) DNA from colorectal cancer patients. The upper and lower bands represent PCR products from amplification of the TSER segment containing 3R and 2R, respectively. The numbers refer to different patient cases. Each patient has a heterozygous 2R/3R genotype in normal tissue, as indicated by the presence of both bands. Case 1: LOH gives rise to a tumor with a 2R/loss genotype. Case 2: LOH does not happen. Case 3: LOH gives rise to a tumor with a loss/3R genotype.

FIG. 2 depicts survival (Kaplan-Meier) plots indicating probability of survival for patients with each of the various possible tumor TSER genotypes separately.

FIG. 3 depicts survival (Kaplan-Meier) plots indicating probability of survival for patients grouped according to 2R genotype only (2R/loss+2R/3R), 3R genotype only (3R/3R+3R/loss) and heterozygotic (2R/3R).

FIG. 4 shows the sequences of the amplification primers and probes for TS mRNA quantitation (Table 1).

FIG. 5 is a table (Table 2) showing the TS genotypes in normal tissue of a study of CRC patients.

FIG. 6 is a table (Table 3) showing the frequency of loss of heterozygosity (LOH) in tumor tissue in a study of CRC patients.

FIG. 7 is a table (Table 4) showing the associations between clinicopathological variables and TS genotype modulated by LOH.

FIG. 8 is a table (Table 5) showing the clinical outcome of patients, segregated by TS genotype.

FIG. 9 is a table (Table 6) showing TS expression, segregated by TS genotype.

### DETAILED DESCRIPTION OF THE INVENTION

The present invention provides a method of predicting a response to a chemotherapeutic regimen based on loss of heterozygosity at the thymidylate synthase locus in cancer tissue.

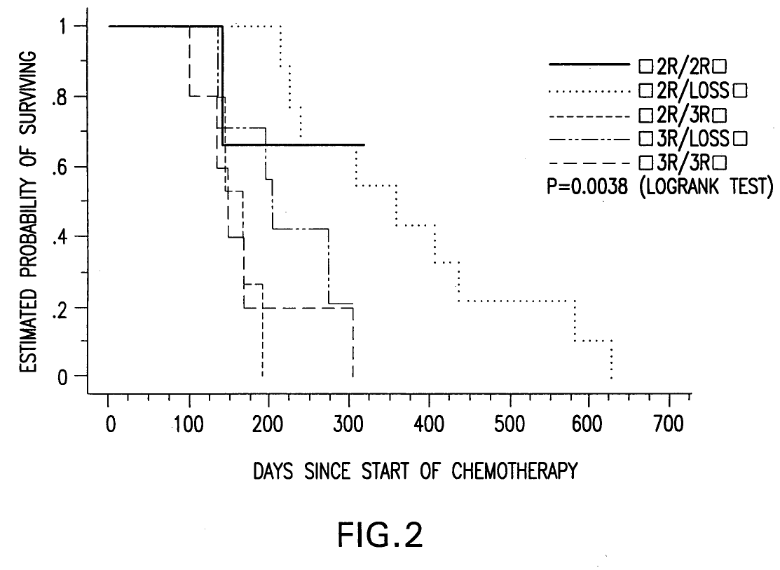

The TS gene has been localized to the telomeric region of the short arm of chromosome 18, at chromosome band 18p11.32 (Hori, T., et al., Human Genetics, 85(6):576-80, 1990), a region of the genome which is known to undergo deletions in a high percentage of colorectal cancers (Vogelstein B., et al., Science, 244:207-211, 1989). One of the main targets of these deletion events may be the well-known tumor suppressor gene DCC (deleted-in-colorectal cancer) (Klingelhutz A J, et al., Oncogene 10: 1581-1586, 1995). Due to its proximity to the target gene(s) of chromosome 18 deletions, the TS gene in some cases may be contained within the deleted DNA segment. Indeed, the inventors confirmed in an earlier study that loss of heterozygosity (LOH) occurred frequently at the TS locus in tumor DNA when analysis of the TS genotype from 2R/3R individuals by gel electrophoresis showed a different ratio between 2R and the 3R bands in tumor tissue samples than in normal tissues (i.e., an allelic imbalance) (Kawakami K, et al., Jpn J Cancer Res., 93: 1221-1228, 2002), arising from the fact that upon LOH, a heterozygous 2R/3R genotype would convert to either a 2R/loss or 3R/loss genotype. In this previous study (Id.) (which is herein incorporated in its entirety be reference), the observed frequency of LOH was 62% (31 of 50) among patients heterozygous for the TS promoter, which is almost identical to that reported by Zinzindohue et al. (19/30; 63%) in their group of colorectal cancer (CRC) patients (Zinzindohoue F, et al., J. Clin. Oncol., 19:3442, 2001). The observed LOH frequency in the set of patients of the present study was even higher (77%), which may be ascribed to the use of laser capture microdissection (LCM) of all specimens to isolate the areas of tumor cells as free as possible of any stromal tissue. With this technique for purifying tumor tissue, it is possible to observe quite unambiguously, as demonstrated in FIG. 2, the loss of one or the other TS allele and thus to show definitively the presence or absence of an LOH event at the TS locus.

The present invention is based in part on the observation that the TS locus has a relatively frequent loss of heterozygosity LOH in cancer tissue, which leads to modification of TS genotype in the tumor when it is heterozygous in normal tissue. That is, LOH in individuals who have a heterozygous 2R/3R genotype in their normal tissue would give rise to a tumor with either a 2R/loss or the 3R/loss genotype, depending on which allele became deleted during the LOH event. Thus, if the 3R allele is lost, the tumor has a 2R/loss genotype. If the 2R allele is lost, the tumor has a 3R/loss genotype.

The present invention is based in part on the discovery that tumors acquire the respective chemosensitivities characteristic of 2R/2R and 3R/3R tumors when LOH at the TS locus in tumors of heterozygous 2R/3R and results in either a 2R/loss or a 3R/loss genotype. The salient finding of this study is that the tumor genotype determines the outcome of the therapy: 2R/3R patients with a 2R/loss genotype in the tumor had the same survival as that patients with only 2R genotypes whereas the 2R/loss genotype had strikingly better results from the treatment than patients with the 3R/loss genotype, both in terms of response rate (80% vs. 14%) and survival (333 days vs. 203 days). These results suggest a direct link between TSER polymorphism status in the tumor tissue and response to a TS inhibitor (such as, but not limited to 5-FU, FUdR, UFT, S-1 and capecitabine) based therapy. The data further illustrate that the triple TSER repeat (3R) has a direct negative effect on tumor response as opposed to 2R having a positive effect. As shown in FIG. 2, the survival times of patients who have the tumor 2R/3R genotype are short, similar to those of the 3R-only patients, whereas if the 3R allele is lost during LOH, the resulting 2R/loss tumor has a high response rate and long survival.

The mechanisms by which TSER polymorphic repeats effect tumor response is still unclear. The observation of Horie et al. (Cell Struct. Funct., 20:191-197,1995) that the expression activity of a reporter gene linked to the TS gene with the double repeat was lower than that of the gene with the triple repeat suggested the possibility that TSER polymorphisms effect tumor sensitivity to 5-FU by regulating TS gene expression. This hypothesis seemed to be confirmed by the results of Pullarkat et al. (Pullarkat, S. T., et al., Pharmacogenomics J., 1, 65-70, 2001), who reported a significant TSER-dependent difference (ca. 3.6-fold) in TS gene expressions in colorectal cancer patients and suggested that this difference accounted for the lower response rates of 3R/3R cancer patients compared to 2R/2R genotypes. However, when other data are considered, the association between the number of TSER polymorphisms and TS gene expression does not appear straightforward. For instance, 130 colorectal cancer specimens were analyzed and no significant difference in TS gene expression between 2R/2R and 3R/3R genotypes was found. Instead 3R/3R tumors had a significantly higher TS protein level (Pullarkat, S. T., et al., Pharmacogenomics J, 1, 65-70, 2001), which would also account for lower response rates among 3R/3R patients. This finding suggested that translational activity producing TS protein from mRNA is effected by TSER polymorphism, as is indeed was supported by in vitro experiments showing that the 3R promoter caused an increase in translation activity (Id.). Etienne et al. analyzed 103 CRC tumors treated with 5-FU/folinic acid and found, interestingly, that 2R/3R tumors had higher TS enzyme activity than either 2R/2R or 3R/3R tumors, as well as the shortest survival of the patients.

In the study leading to the present invention, tumors with only 3R genotypes (3R/3R+3R/loss) showed higher mean TS gene expression than those with only 2R genotypes (˜1.5-fold). The finding that 3R/loss genotypes had higher expression than the 2R/loss genotypes (Table 6) by a similar amount suggests that the 3R does exert a direct, albeit modest, up-regulation of TS gene expression. However, the small difference in mean TS gene expression between tumors bearing only 2R or 3R genotypes seems insufficient to account for the rather striking difference in response to S-1 treatment, suggesting that mechanisms other than (or in addition to) TS regulation may be involved.

For example, several recent studies suggest that TSER repeats have a role in regulating the levels of folate metabolites, which could affect drug toxicity to patients and anti-tumor activity of drugs that interact with folate-utilizing enzymes. Significantly lower levels of plasma folates and homocysteine were found in 3R/3R genotype individuals (Trinh, B. N., et al., Hum. Genet., 111:299-302, 2002). This observation might account for the lower toxicity to 5-FU treatment experienced by 3R/3R patients (Pullarkat, S. T., et al., Pharmacogenomics J, 1, 65-70, 2001) because elevated levels of plasma homocysteine have been found to be associated with higher risk of drug toxicity (Niyikiza, C., et al., Molecular Cancer Therapeutics, 1:545-52, 2002). TSER repeat status has been linked to response of adult acute lymphoblastic leukemia (ALL) to methotrexate, a dihydrofolate reductase inhibitor, which may also be influenced by folate levels (Krajinovic, M., et al., Lancet, 359: 1033-1034, 2002.). Associations have been noted among TSER polymorphisms, folate intake and risk of colorectal cancer (Ulrich, C. M., et al., Cancer Res., 62: 3361-3364, 2002) as well as with overall risk of developing risk of adult ALL. (Skibola, C. F., et al., Blood, 99:3786-91, 2002).

Thus, the present invention is based in part on the discovery that a high percentage of cancer patients undergo LOH at the TS locus in the tumor and those who have a 2R/3R normal tissue genotype segregate into two groups with different tumor TS genotypes, one of whom (the 2R/loss patient) can anticipate considerably better clinical outcome from fluoropyrimidine treatment than the other (the 3R/loss patient). Whether a particular individual will acquire a 2R/loss or a 3R/loss tumor is a matter of chance with an equal probability of ending up with either one. Thus, analysis of TS genotype in tumor tissue, in conjunction with TS gene expression measurements, can help to identify patients who should be considered as good candidates for chemotherapy with TS-directed regimens and those who, due to their lower probability of response, should be considered for another type of treatment.

Thus, the present invention relates to the use of these TS gene polymorphisms as tools for prediction of response and selection of therapy. It is therefore evident that analysis only of normal tissue is insufficient but tumor tissue must also be analyzed to establish the TS polymorphism status in the tumor. In addition, the present invention also contemplates anaylzing normal tissue genotype as it also provides insight into predicting a response to a chemotherapeutic regimen, particularly in predicting drug toxicity levels.

Thus, in predicting a response to a chemotherapeutic regimen the genotype of the tumor tissue is relevant in predicting whether the tumor is likely to respond to a chemotherapeutic regimen. Further the genotype of the non-tumor tissue may be relevant in predicting the risk of developing drug toxicity. Thus, the knowledge of both tissue genotypes assists the physician in developing a suitable chemotherapeutic regimen that will have a greater chance of success in battling the tumor, while also decreasing the risk of drug toxicity.

Accordingly, one aspect of the present invention provides a method of predicting a response to a chemotherapeutic regimen based on loss of heterozygosity at the thymidylate synthase locus in cancer tissue. This method comprises determining the genotype for thymidylate synthase of a patient in normal tissue and in the cancerous tissue. The two genotypes are compared to determine whether a loss of heterozygosity at the thymidylate synthase locus has occurred in the tumor tissue. Based on the loss of heterozygosity in the tumor sample, a response to a chemotherapeutic regimen can be predicted. As shown above, a patient having a 2R/loss genotype in the tumor sample will likely have a similar response to the chemotherapeutic regimen as patients having a 2R/2R tumor sample genotype. For example, if patients having a 2R/2R tumor sample respond positively to a chemotherapeutic regimen, then it can be predicted that patients having a 2R/loss tumor genotype will also respond positively to the same chemotherapeutic regimen. Similarly, if patients having a 3R/3R tumor sample respond negatively or show no response to a chemotherapeutic regimen, then it can be predicted that patients having a 2R/loss tumor genotype will also respond negatively or show no response to the same chemotherapeutic regimen.

Further, the genotype of the normal tissue may be used to predict the patients risk in developing drug toxicity. For example, as stated above studies have shown that 3R/3R patients were found to experience lower risks of drug toxicity than 2R/2R patients. Thus, a 2R/3R normal tissue patient will likely exhibit drug toxicity at doses somewhat lower than 3R/3R patients, but will likely be able to withstand higher doses than 2R/2R patients.

What is meant by the term “response” relates the effects of a chemotherapeutic regimen on tumor tissue or cells. Methods of measuring such response are defined with varying criteria as appropriate, usually depending upon the chemotherapeutic regimen and the type of cancer being treated. Generally, criteria include tumor size reduction, distant site metastases (such as lymph node and lung metastases in the case of colorectal cancer) and others depending upon the cancer tissue type, stage of cancer and metastases and chemotherapeutic regimen. One skilled in the art would appreciate and understand the appropriate criteria for the conditions surrounding the tumor type and the chemotherapeutic regimen. For example, in one of the studies leading to the present invention, to be classified as a “responder” to a chemotherapeutic regimen, a tumor had to have a 50% reduction in the sum of the products of the perpendicular diameters of the indicator lesion without growth of other disease or the appearance of new lesions. Thus, a positive response in this case would mean tumor reduction and no formation of new lesions. Conversely, a negative response would mean no tumor reduction or even continued tumor growth and/or the appearance of new lesions.

In one embodiment of the present invention the genotype of a patient's normal tissue is also used to predict potential drug toxicity. For example, as stated above studies have shown that 3R/3R patients were found to experience lower risks of drug toxicity to 5-FU than 2R/2R patients. Thus, a 2R/3R normal tissue patient will likely exhibit drug toxicity at doses somewhat lower than 3R/3R patients, but will likely be able to withstand higher doses than 2R/2R patients. Thus, using one of the methods of the present invention to predict a response to a chemotherapeutic regimen, one would predict a patient having a 2R/3R normal tissue genotype and a 2R/loss tumor tissue genotype to react positively to 5-FU treatment (i.e. decrease in tumor size), but be more sensitive to drug toxicity than a patient having a 3R/3R normal tissue genotype. Thus using this prediction, along with compiled data on other similar individuals, a physician could tailor the chemotherapeutic regimen and dosing based on prior patient data relating to tumor responses and drug toxicity.

In determining the genotype of the tumor sample, either fresh or preserved tumor tissue/cells may be used. If preserved, they are preferably preserved as formalin-fixed paraffin embedded (FPE) samples. The FPE tumor sample is then preferably subjected to laser capture micro-dissection (LCM). LCM is useful in isolating tumor cells from non-tumor cells and thus provides a more accurate assessment of tumor tissue genotype.

TS gene expression levels in the tumor tissue may also be measured. Such measurement may assist in predicting a response to a chemotherapeutic regimen as in vitro studies have shown that cells become resistant to TS inhibitors by up-regulating TS expression and raising intracellular TS levels. Studies have shown that TS expression varies considerably among tumors and that sensitivity of various tumors to 5-FU-based chemotherapy is correlated wit the intratumoral level of TS. Thus, in addition to determining the tumor genotype, measuring TS gene expression in the tumor may provide useful information in predicting a response to a chemothereapeutic outcome.

To assist in predicting a response, the present invention also contemplates, compiling data regarding patients' tumor and normal tissue genotype and their responses to chemotherapeutic regimens and/or TS gene expression levels in tumor cells. For example, patients whose tumors responded to a chemotherapeutic regimen may be classified as “responders.” The genotype of their tumor, as well as TS gene expression levels in their tumors is determined and recorded. Likewise, patients whose tumors did not respond favorably to a chemotherapeutic regimine may be classified as “non-responders.” The genotype of their tumor, as well as TS gene expression levels in their tumors is determined and recorded. Then, when a patient is diagnosed with a cancer, the genotype of the tumor as TS gene expression levels in the tumor is tested and compared to the compiled data. If the patient's tumor genotype and TS gene expression levels correlate with the responder group, a positive response may be predicted for that patient. Conversely, when a patient's tumor genotype and TS gene expression levels correlate to the non-responder group, a negative response may be predicted for the chemotherapeutic regimen for that patient.

For example, if a patient “A” has the normal tissue genotype of 2R/3R and the tumor tissue genotype is 2R/loss, using a method of the present invention, one would predict the response to the chemotherapeutic regimen in patient “A” to be similar to a response to the same chemotherapeutic regimen in a patient having a 2R/2R tumor or a 2R/2R normal tissue genotype. As another example, if a patient “B” has the normal tissue genotype of 2R/3R and the tumor tissue genotype is 3R/loss, using a method of the present invention one would predict the response to the chemotherapeutic regimen in patient “B” to be similar to a response to a same chemotherapeutic regimen in patients having a 3R/3R cancer or normal tissue genotype.

Similarly, in predicting drug toxicity, the present invention contemplates compiling data regarding drug toxicity levels in patients, along with the patients' normal tissue and tumor tissue genotype. Thus, a “safe” level of drug administered (before causing lethal drug toxicity) may be predicted based on the patient's normal tissue genotype in comparison with other patients having the same normal tissue genotype and their safe drug dosage levels.

The chemotherapeutic regimen may be any regimen useful in treating the cancer/tumor at issue. One skilled in the art will be knowledgeable in the appropriate chemotherapeutic regimen to choose. Various factors include, but are not limited to, the type of cancer, the patient's status/health, the extent of the cancer, tumor size, etc. In addition, other therapies may also be used simultaneously such as radiation therapy or a chemotherapeutic drug combination. Also the chemotherapeutic regimen may combine other therapies including the use of gene therapies using antisense oligonucleotides, cancer antigen-directed antibodies, co-factors, radiation treatment and the like.

In the case of human gastrointestinal cancers, such as colorectal cancer, TS inhibitors including fluoropyrimidines and folate-based inhibitors, seem to be the current in vogue chemotherapies. Thus, the present invention contemplates predicting a response to, but not limited to, TS inhibitors. TS inhibitors include, but are not limited to 5′-FU, FUdR, UFT, S-1, capecitabine, ratitrexed, pemetrexed, and nolatrexed, or a combination thereof. Often in treating CRC, multiple therapies are involved. These include the administration of additional chemotherapeutic substances, such as cisplatin, oxaliplatin, taxanes as well as radiation treatment.

### EXAMPLES

### Example 1

Patient Population

Eligible patients had (a) a diagnosis of disseminated or recurrent colorectal cancer after surgical operation; (b) a Eastern Cooperative Oncology Group performance status of 0 to 2 with adequate hematological, hepatic, and renal function; (c) no treatment during the preceding four weeks; and (d) a lesion that was measurable by radiological examination.

Treatment

Patients were treated with S-1 twice daily for 28 days, followed by a 2-week period of rest. The S-1 was given orally after breakfast and dinner. As in previous phase II studies (Villafranca, E., et al., J. Clin. Oncol., 19: 1779-1786, 2001; Zinzindohoue, F., et al., J. Clin. Oncol., 19:3442, 2001.), Body surface area (BSA) was used to determine the dose of S-1 administered, as follows: BSA<1.25 m², 40 mg; 1.25-1.5 m², 50 mg; >=1.5 m², 60 mg. These treatments were repeated until disease progression as determined by the treating physician, or at the physician's discretion.

The protocol was reviewed and approved by an institutional review board and an ethics committee before study activation, and informed consent was obtained from every patient according to the institutional regulations.

Evaluation

After two cycles of treatment, measurable disease was reassessed. Response criteria were the standard definitions used for national cooperative group trials (Green, S. et al., Investig. New. Drugs, 10: 239-253, 1992). Response was assessed by CT in liver, lymph node, and lung metastases, as well as in primary lesions. To be classified as a responder, a tumor had to have a 50% reduction in the sum of the products of the perpendicular diameters of the of the indicator lesion without growth of other disease or the appearance of new lesions (Id.).

Microdissection

A representative formalin-fixed, paraffin-embedded pre-S1 treatment tumor specimen was selected by a pathologist after examination of the hematoxylin and cosin stained slides. Ten micron thick sections were stained with neutral fast red to enable visualization of histology for laser capture microdissection (P.A.L.M. Microlaser Technologies AG, Munich Germany), which was performed to ensure that only tumor cells were studied. Tissue collected from the specimen that had no cancer invasion by histopathology was considered to be normal tissue.

RNA Isolation and cDNA Synthesis

RNA isolation from paraffin embedded specimens was done according to the procedure set out in U.S. Pat. No. 6,248,535, which is hereby incorporated by reference in its entirety. Following RNA isolation, cDNA was prepared from each sample as described previously (Lord R V, et al., J. Gastrointest Surg., 4: 135-142, 2000).

RT-PCR

Relative cDNA quantitation for TS and an internal reference gene (β-actin) was done using a fluorescence based real-time detection method (ABI PRISM 7900 Sequence Detection System [TaqMan®], Applied Biosystems, Foster City, Calif.), as described previously (Lord R V, et al., J. Gastrointest Surg., 4: 135-142, 2000; Heid C A, et al., Genome Res., 6: 986-994, 1996; Gibson U E, et al., Genome Res., 6: 995-1001, 1996). The primers and probe sequences used are given in Table 1. The PCR reaction mixture consisted of 600 nM of each primer, 200 nM probe, 2.5 U AmpliTaq Gold Polymerase, 200 μM each dATP, dCTP, dGTP, 400 μM dUTP, 5.5 mM MgCl₂, and 1×Taqman Buffer A containing a reference dye, to a final volume of 25 μl (all reagents Applied Biosystems, Foster City, Calif.). Cycling conditions were 50° C. for 10s, 95° C. for 10 min, followed by 46 cycles at 95° C. for 15s and 60° C. for 1 min. Colon, liver, and lung RNAs (all Stratagene, La Jolla, Calif.) were used as control calibrators on each plate.

DNA Extraction, PCR and Electrophoresis

Collected DNA was extracted using the QIAamp Kit (Qiagen, Valencia, Calif., USA). The promoter region of the TS gene was amplified by polymerase chain reaction (PCR) using the following primers: forward primer 5′-GCGGAAGGGGTCCTGCCA-3′ and reverse primer 5′-TCCGAGCCGGCCACAGGCAT-3′. PCR was performed using the conditions described previously (Kawakami, K., et al., Anticancer Res., 19: 3249-3252, 1999). The PCR products was analyzed by electrophoresis on a 10% TBE-Urea Polyacrylamide gel (Invitrogen Corp., Carlsbad, Calif.).

Statistical Analysis

The gene expression values are expressed as ratios between two absolute measurements (gene of interest/internal reference gene). The gene expression values in each TS genotype group were analyzed using the Mann-Whitney U test. Chi-square for independence test was used to assess the association between TS genotype and response to chemotherapy. The log-rank test was used to measure the association between TS genotype and survival.

Results

A total of 30 primary pre-S1 treatment colorectal cancer specimens from 30 patients, all of whom had Stage 1V disease, were studied. Thirteen of the patients (43.3%) were classified as responders to S-1 and 17 patients (56.7%) were non-responders. TS genotype and TS expressions values were obtained for all of 30 patients. Fourteen of the 30 patients (46.6%) included in the study were male and the median age was 65.0 (range 39-79). The median overall survival time for all 30 patients was 215.5 days (range 98-627). The median overall survival for patients with S-1 responding tumors was 303 days (range 139-627) and for non-responders was 190 days (range 98-435).

TS polymorphism and LOH in the Number of TS Repeat Sequence in Colorectal Normal Tissue and Cancer Tissue

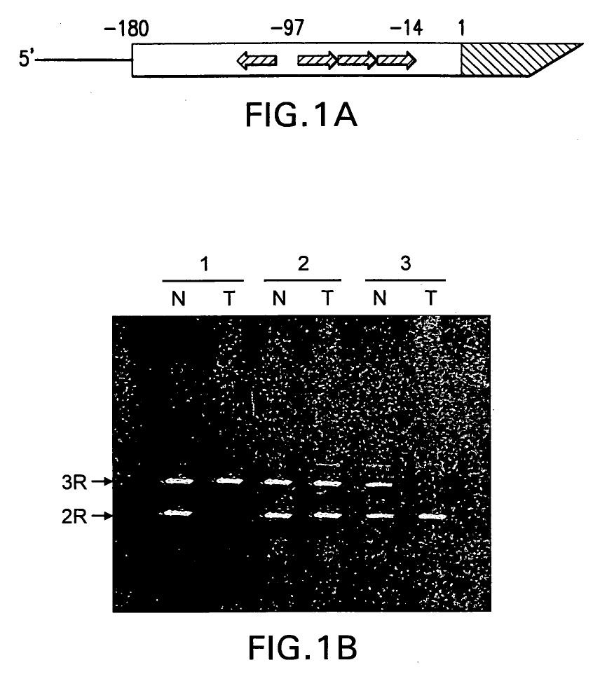

PCR fragments with estimated length of 107 and 135 bp (FIG. 1) were obtained. The 107- and 135-bp fragments represent the two- and three-repeat (2R and 3R) sequences. The TS genotypes were classified into 2R-homozygote (2R/2R), 3R-homozygote (3R/3R), and 2R/3R-heterozygote. The frequency of each genotype in the 30 colorectal normal tissues is shown in Table 2. In the 22 normal tissues with the 2R/3R genotype, the 10 cancer tissues showed only 2R-sequence band (2R/loss), and the 7 cancer tissues showed only 3R-sequence band (3R/loss). See Table 3 and FIG. 1. The each rate of incidence of loss of heterozygosity in the TS locus is. 45% (10/22) of 2R/loss genotype and 31.8% (7/22) of loss/3R genotype (Table 3).

Clinicopathological Characteristics and TS polymorphism Modulated by LOH

The 30 patients were divided into five groups depending on TS genotype modulated by LOH. Regarding clinicopathological characteristics, there was no significant difference between these five groups (Table 4). Regarding relapse category, 13 of the all 30 patients (43%) had liver metastasis. The majority of tumors (29/30) were well or moderately differentiated adenocarcinoma histologically.

TS Polymorphism Modulated by LOH and Response to S-1 Chemotherapy

The response rate of the 10 patients with 2R/loss genotype in the cancer is 80% (8/10) and highest in the each genotypes, and the response rate of the 7 patients with 3R/loss genotype is 14% (7/22) and lowest in the each genotypes. There is a significant difference between each response rate (p=0.029) (Table 5).

TS Polymorphism Modulated by LOH and Overall Survival

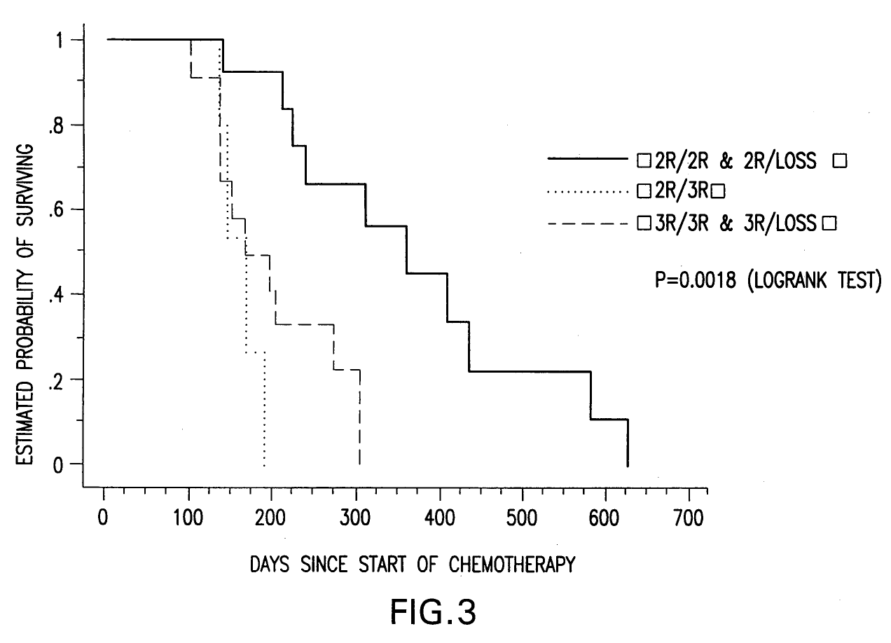

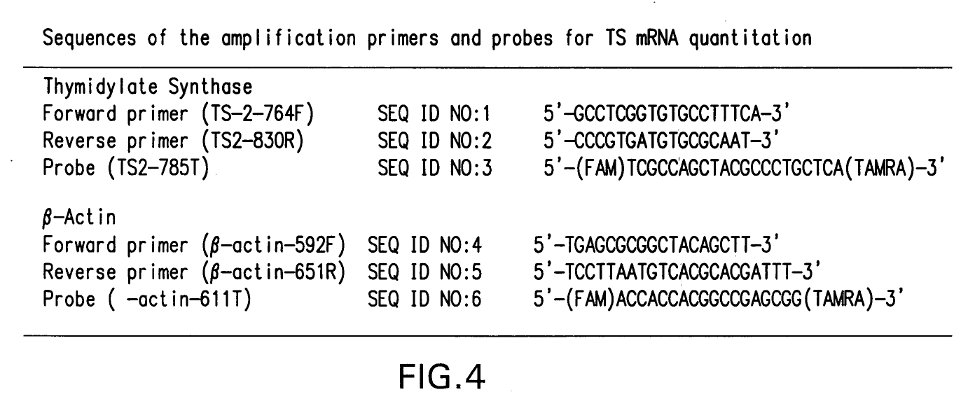

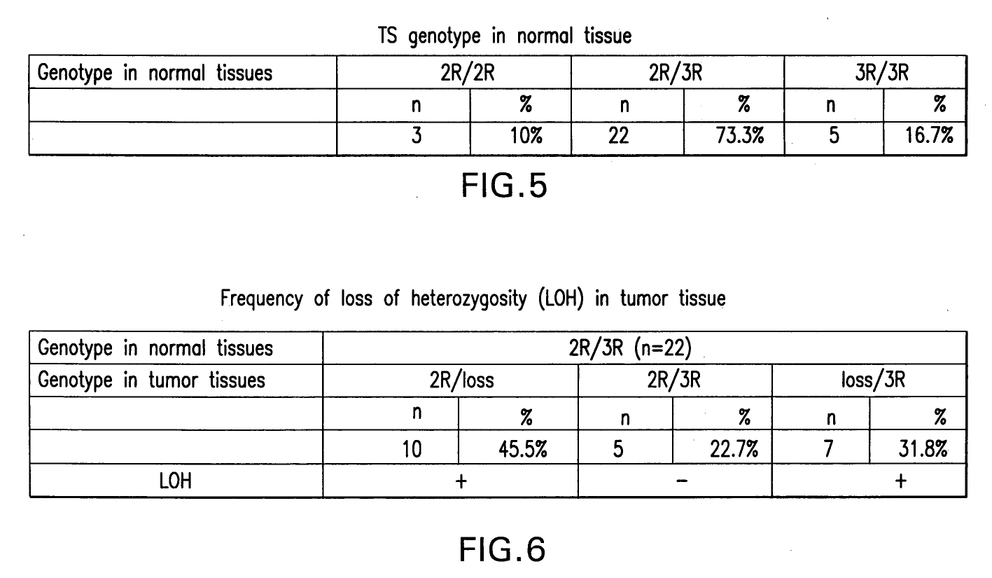

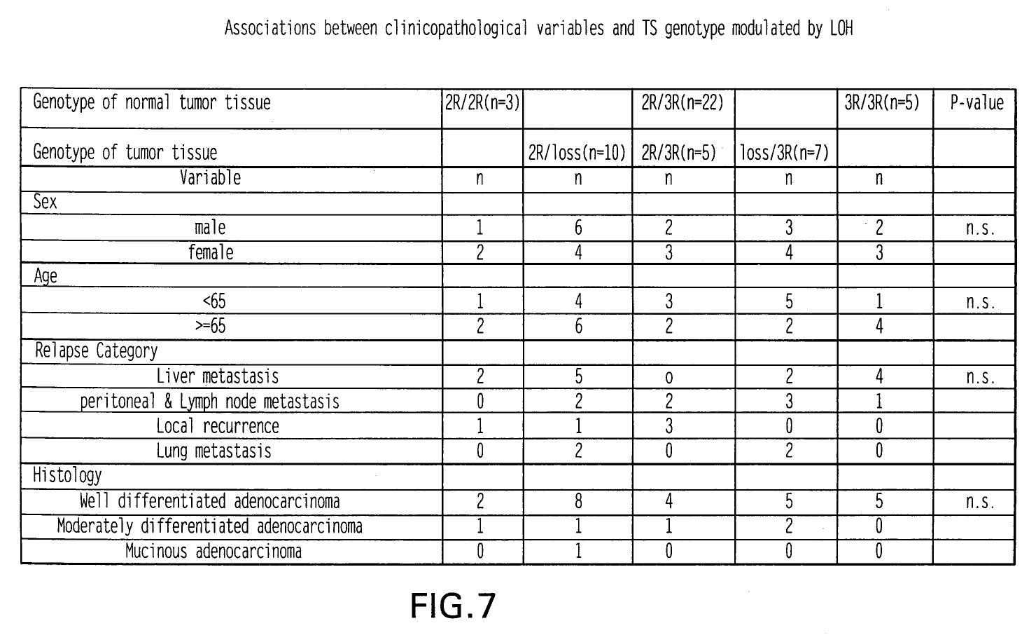

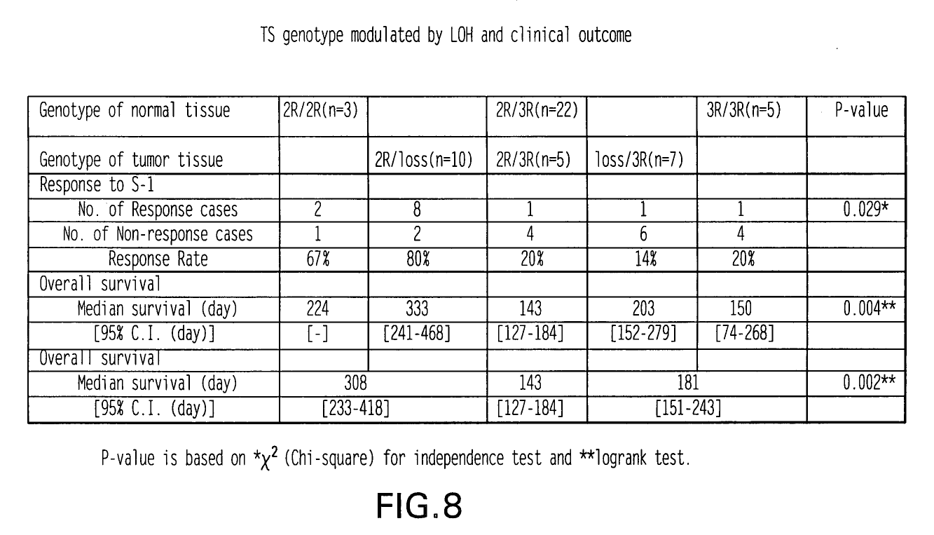

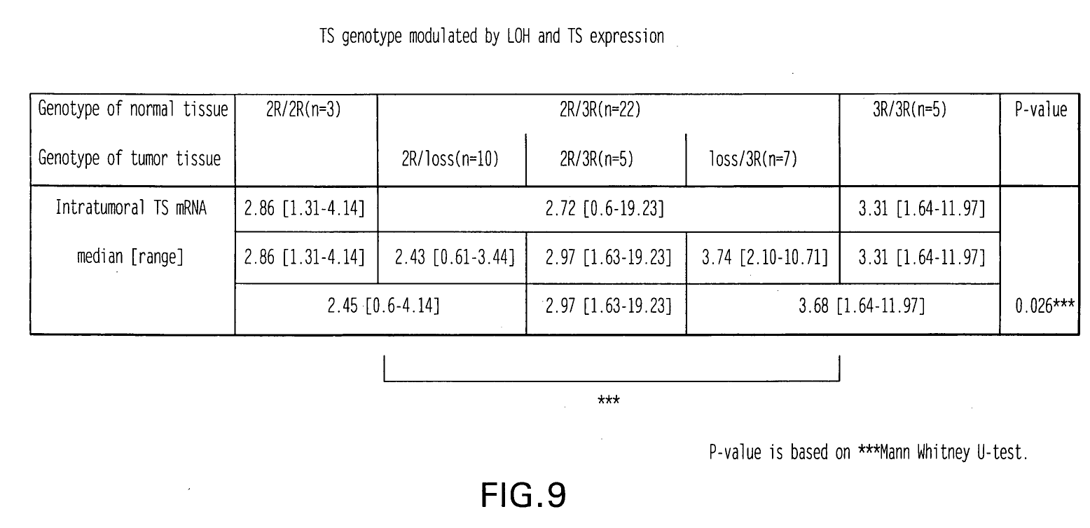

The median overall survival periods were 333 days (95% C.I; 241-468 d.) for those colorectal patients with 2R/3R genotype in their normal tissue and 2R/loss genotype in cancer tissue. This survival is significantly longer than that of patients with other genotypes (p=0.004) (Table 4 and FIG. 2). The median overall survival periods were 308 days (95% C.I; 233418 d.) for colorectal patients with 2R/2R or 2R/loss genotype in the cancer tissue. This is significantly longer than that of patients with other genotypes in the cancer tissue (p=0.002) (Table 5 and FIG. 3).

TS Polymorphism Modulated by LOH and Intratumoral TS mRNA Expression

The median intratumoral TS mRNA expression were 2.45 (range 0.6-4.14) for patients with 2R/2R or 2R/loss genotype in the cancer tissue, 2.97 (range 1.63-19.23) for patients with 2R/3R genotype in the cancer tissue, and 3.68 (range 1.64-11.97) for patients with loss/3R or 3R/3R genotype. The TS genotype modulated by LOH in the cancer tissue was statistically associated with intratumoral TS mRNA expression (p=0.026) (Table 6).

1) a method of predicting a response to a chemotherapeutic regimen based on loss of heterozygosity at the thymidylate synthase locus in cancer tissue, said method comprising:
(a) determining normal tissue genotype for thymidylate synthase of a patient;
(b) determining tumor tissue genotype for thymidylate synthase of said patient;
(c) comparing the normal tissue genotype with the tumor tissue genotype;
(d) determining whether a loss of heterozygosity at the thymidylate synthase locus has occurred in the tumor tissue based on the comparison of step (c);
(e) predicting a response to the chemotherapeutic regimen based on the loss of heterozygosity in the tumor sample.

2) The method of claim 1, wherein the tumor tissue is formalin-fixed paraffin embedded.

3) The method of claim 2, further comprising subjecting tumor tissue from formalin-fixed, paraffin embedded (FPE) to laser capture micro-dissection.

4) The method of claim 3 further comprising measuring TS gene expression levels in the tumor tissue.

5) The method of claim 4, further comprising
(i) determining levels of TS gene expression in tumor cells of responder patients, said responder patients having tumors that responded to the chemotherapuetic regimen;
(ii) determining levels of TS gene expression in tumor cells of non-responder patients, said non-responder patients having tumors that showed no response to the chemotherapeutic regimen;
(iii) comparing levels of TS gene expression in tumor tissue of said patient with TS gene expression levels of responder and non responder patients;
(iv) predicting a response to the chemotherapeutic regimen based on the comparison of step (iii).

6) The method of claim 1 wherein the normal tissue genotype is 2R/3R and the tumor tissue genotype is 2R/loss and wherein the predicting the response to the chemotherapeutic regimen is predicted to be similar to a response to a same chemotherapeutic regimen in a patient having a 2R/2R cancer or normal tissue genotype.

7) The method of claim 1 wherein the normal tissue genotype is 2R/3R and the tumor tissue genotype is 3R/loss, and wherein the predicting the response to the chemotherapeutic regimen is predicted to be similar to a response to a same chemotherapeutic regimen in a patient having a 3R/3R cancer or normal tissue genotype.

8) The method of claim 1 wherein the chemotherapeutic regimen comprises administration of a TS inhibitor.

9) The method of claim 8 wherein the chemotherapeutic regimen comprises administration of 5′-FU, FUdR, UFT, S-1, capecitabine, ratitrexed, pemetrexed, or nolatrexed, or a combination thereof.

10) The method of claim 9 wherein the chemotherapeutic regimen comprises administration of 5′-FU, FUdR, UFT, S-1, capecitabine, ratitrexed, pemetrexed, or nolatrexed in combination with cisplatin, oxaliplatin, taxanes or radiation.

11) The method of claim 1 further comprising comparing the patient's normal tissue genotype with other individuals having a normal tissue genotype the same as said patient;
(i) calculating drug toxicity levels in said other individuals having been treated with a chemotherapeutic regimen; and
(ii) correlating said drug toxicity levels to said patient to predict the patient's response to the same chemotherapeutic regimen.


SEQUENCE LISTING (6 sequences)

SEQ ID NO: 1 - DNA, 18 bp, Artificial Sequence
  Description of Artificial Sequence Primer
gcctcggtgtgcctttca

SEQ ID NO: 2 - DNA, 17 bp, Artificial Sequence
  Description of Artificial Sequence Primer
cccgtgatgtgcgcaat

SEQ ID NO: 3 - DNA, 21 bp, Artificial Sequence
  Description of Artificial Sequence Probe
tcgccagctacgccctgctca

SEQ ID NO: 4 - DNA, 18 bp, Artificial Sequence
  Description of Artificial Sequence Primer
tgagcgcggctacagctt

SEQ ID NO: 5 - DNA, 22 bp, Artificial Sequence
  Description of Artificial Sequence Primer
tccttaatgtcacgcacgattt

SEQ ID NO: 6 - DNA, 18 bp, Artificial Sequence
  Description of Artificial Sequence Probe
accaccacggccgagcgg